(19) United States
(12) Patent Application Publication
(10) Pub. No.: US 2025/0000604 A1
(43) Pub. Date: Jan. 2, 2025

(54) SCREEN TILT ASSEMBLY

(71) Applicant: Sunset IP Pty Ltd, Banyo (AU)

(72) Inventors: Matthew Cramsie, Banyo (AU); Murray Ian Davidson, Banyo (AU)

(21) Appl. No.: 18/759,056

(22) Filed: Jun. 28, 2024

(30) Foreign Application Priority Data
Jun. 30, 2023 (AU) 2023902097

(51) Int. Cl. A61B 90/00; A61B 50/13; F16M 11/06

(52) U.S. Cl. CPC A61B 90/37; A61B 50/13; F16M 11/06; F16M 2200/025

(57) ABSTRACT

A screen tilt assembly for use with medical carts, the screen tilt assembly comprising a bracket member comprising at least two spaced apart connection plates arranged in a plane for affixing a screen thereto, the at least two spaced apart connection plates joined by an arcuate portion extending away from the plane; and a mounting arrangement for connection to a medical cart, the mount arrangement comprising a first clamping member and a second clamping member configured for selectively clamping a section of the arcuate portion of the bracket member therebetween to retain the bracket member in place relative to the mounting arrangement; wherein the arcuate portion is configured to be selectively moved relative to the mounting arrangement thereby tilting the plane.

### TECHNICAL FIELD

The present invention relates to a screen tilt assembly for use with medical carts.

### BACKGROUND

Any references to methods, apparatus or documents of the prior art are not to be taken as constituting any evidence or admission that they formed, or form part of the common general knowledge.

Medical professionals' range in height and have varying preferences for their viewing of medical monitors and screens. Accordingly, there is a need for devices which allow adjustments to medical screens and monitors without sacrificing the space required to provide such a mechanism and/or adjust to a preference or comfortable viewing angle.

As medical equipment improves and the need for specialist medical attention continues to grow, space within medical facilities becomes a much-needed commodity. New machines and devices seek to provide quicker and more accurate information for patients. Innovations which assist in maximising the utility of space without sacrificing function are sought.

### SUMMARY OF INVENTION

In an aspect, the invention provides a screen tilt assembly for use with medical carts, the screen tilt assembly comprising:
a bracket member comprising at least two spaced apart connection plates arranged in a plane for affixing a screen thereto, the at least two spaced apart connection plates joined by an arcuate portion extending away from the plane; and
a mounting arrangement for connection to a medical cart, the mount arrangement comprising a first clamping member and a second clamping member configured for selectively clamping a section of the arcuate portion of the bracket member therebetween to retain the bracket member in place relative to the mounting arrangement;
wherein the arcuate portion is configured to be selectively moved relative to the mounting arrangement thereby tilting the plane.

In an embodiment, the arcuate portion extends in a direction substantially orthogonal to the plane.

In an embodiment, the first clamping member and the second clamping member comprise complementary arcuate profiles which conform to the receive therebetween the arcuate portion of the bracket member.

In an embodiment, the arcuate member comprises one of a slot or a protrusion and the first clamping member comprises the other one of the slot or the protrusion.

In an embodiment, the slot and the protrusion intersect to limit the range of motion of the bracket member relative to the mounting arrangement.

In an embodiment, each of the connection plates comprise one or more apertures.

In an embodiment, the one or more apertures through each of the connection plates align with one or more threaded holes of a screen.

In an embodiment, the mounting arrangement comprises a clamping mechanism for clamping the first clamping mechanism towards the second clamping mechanism.

In an embodiment, the clamping mechanism comprises a bolt and nut arrangement.

In an embodiment, a bolt head for adjusting the bolt and nut arrangement is accessible from an upper portion of the first clamping member or the second clamping member.

In another aspect, the invention provides a method for adjusting a screen tilt assembly, the method comprising:
providing a screen tilt assembly having a bracket member comprising at least two spaced apart connection plates arranged in a plane for affixing a screen thereto, the at least two spaced apart connection plates joined by an arcuate portion extending away from the plane and a mounting arrangement for connection to a medical cart, the mounting arrangement comprising a first clamping member and a second clamping member configured for selectively clamping a section of the arcuate portion of the bracket member therebetween to retain the bracket member in place relative to the mounting arrangement;
actuating a clamping mechanism to allow the mechanism to move the bracket member relative to the mounting arrangement to a desired orientation and actuating the clamping mechanism to lock the bracket member relative to the mounting arrangement.

In an embodiment, the method further comprises loosening a bolt of the clamping mechanism to unclamp the first clamping member from the second clamping member.

In an embodiment, the method further comprises tightening the bolt of the clamping mechanism to clamp the first clamping member to or towards the second clamping member.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is an isometric view of a screen tilt assembly according to an embodiment of the present invention.

FIG. 2 is an isometric view of the bracket member of the screen tilt assembly of FIG. 1.

FIG. 3 is an exploded isometric view of the clamping members of the mounting arrangement of the screen tilt assembly of FIG. 1.

FIG. 4 is an isometric view of a first or second clamping member of the mounting arrangement of the screen tilting assembly of FIG. 1.

FIG. 5 is an isometric view of the other of the first or second clamping member of the mounting arrangement of the screen tilting assembly of FIG. 1.

FIG. 6 is a top view of the screen tilt assembly of FIG. 1.

### DETAILED DESCRIPTION OF PREFERRED EMBODIMENTS

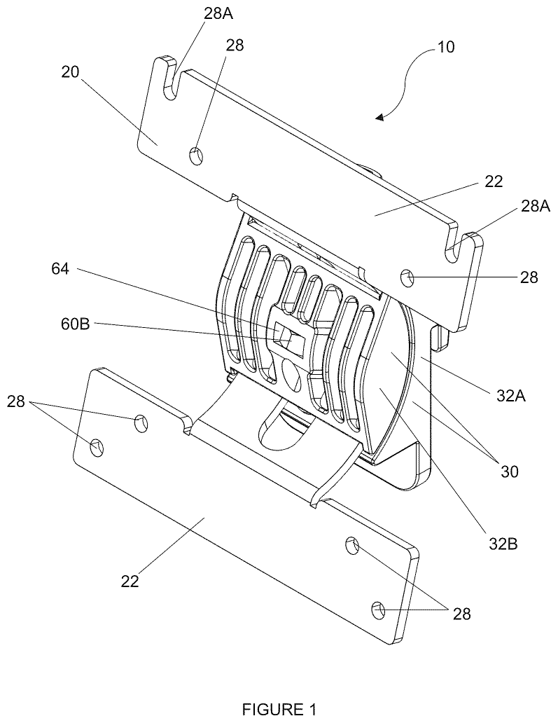

FIG. 1 illustrates a screen tilt assembly 10 according to a preferred embodiment of the present invention. The screen tilt assembly 10 is preferably for use with medical carts which incorporate or house electronic components including monitors and/or screens however it may also have particular benefits in settings where space is a limitation. The screen tilt assembly 10 comprises a bracket member 20 which is selectively movable relative to a mounting arrangement 30 comprising a first clamping member 32A and second clamping member 32B.

Throughout the specification the first clamping member 32A may be referred to as a rear clamping member and the second clamping member 32B may be referred to as a frontal clamping member. The terms rear and frontal are based on the orientation of the bracket member 20 and a prospective screen to be attached. Directional and relational terms are used with respect to the Figures as provided. The person skilled in the art would not infer limitations, express or implied, by the use of these words.

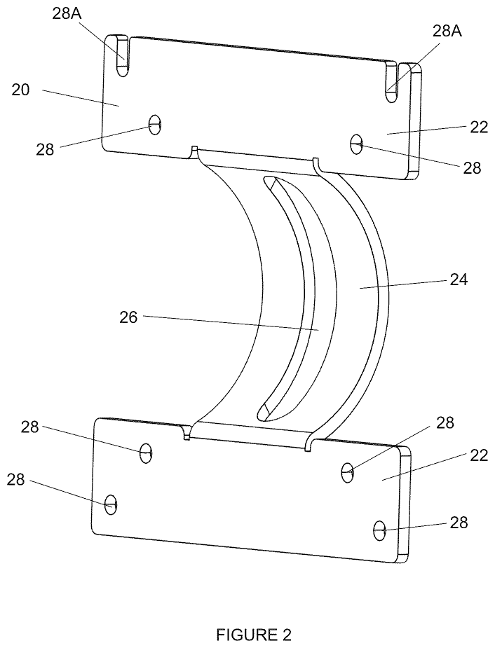

FIG. 2 illustrates the bracket member 20 comprising at least two spaced apart connection plates 22. The at least two spaced apart connection plates 22 are arranged substantially in the same plane. The at least two spaced apart connection plates 22 are configured for affixing a screen or monitor thereto. Preferably, the at least two spaced apart connection plates 22 conform to a rear profile of the screen or monitor to be affixed. Accordingly, in some alternative embodiments, the at least to spaced apart connection plates 22 may slightly curve to the profile of the monitor. Furthermore, in other alternative embodiments, the at least two spaced apart connection plates 22 may comprise separate connection plates for each connection point on the prospective screen. This will be discussed in further detail below.

The at least two spaced apart connection plates 22 are arranged and connected or joined by an arcuate portion 24. The arcuate portion 24 extends away from the plane in which the connection plates 22 are arranged. In the preferred embodiment, the arcuate portion 24 extends generally in a direction that is substantially orthogonal to the plane.

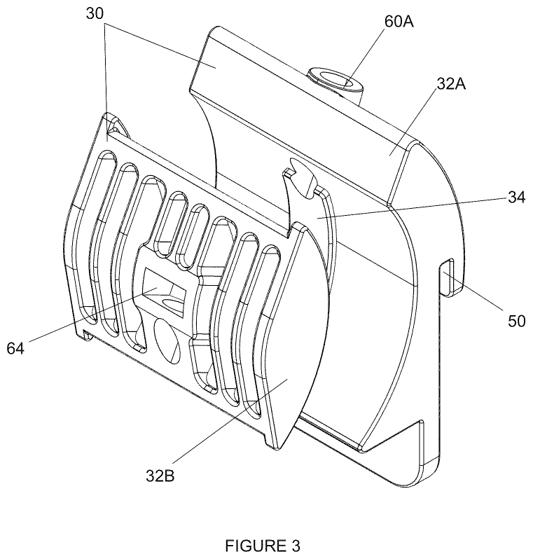

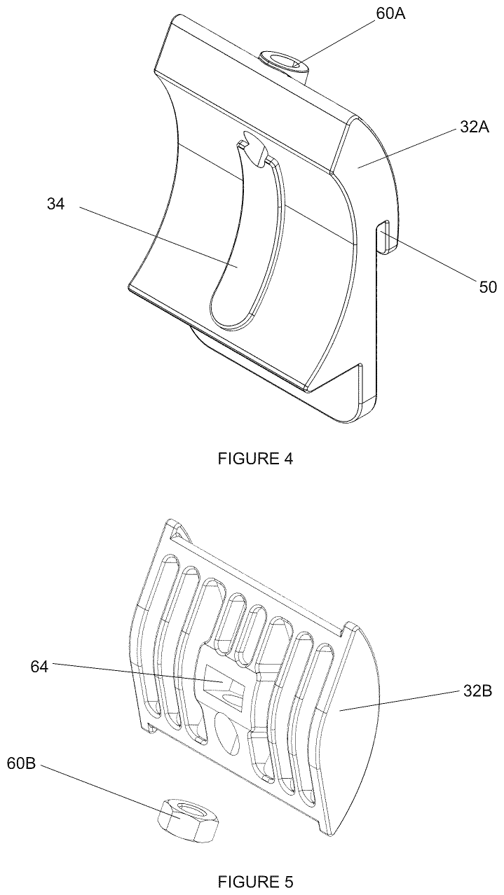

FIGS. 3 to 5 illustrate the mounting arrangement 30. The mounting arrangement comprises a first clamping member 32A or rear clamping member, as seen in FIG. 4, and a second clamping member 32B or frontal clamping member, as seen in FIG. 5. The first clamping member 32A and the second clamping member 32B comprises complementary profiles for clamping a section of the arcuate portion 24 of the bracket member 20 therebetween. A user of the screen tilt assembly can selectively clamp/unclamp the first clamping member 32A and the second clamping member 32B so as to selectively tilt the screen affixed to the screen tilt assembly 10. When unclamped between the first clamping member 32A and the second clamping member 32B, the bracket member 20 is free to rotate relative to the mounting arrangement 30. When clamped between the first clamping member 32A and the second clamping member 32B the bracket member 20 is retained in place relative to the mounting arrangement 30.

The mounting arrangement 30 is configured for connection to a medical cart (not shown). As seen in FIGS. 3 and 4, the rear clamping member or first clamping member 32A comprises a recess 50 which is suitable for receiving a post of a medical cart to connect the screen tilt assembly 10 thereto. The person skilled in the art would readily appreciate there are a number of ways in which the screen tilt assembly 10 may be connected to the medical cart including but not limited to frictional engagement, threaded rods, etc.

As best seen between FIGS. 2 and 4, the bracket member 20 comprises a slot 26 and the mounting arrangement 30 comprises a projection 34. When assembled, the slot 26 receives and is intersected by the projection 34. The projection 34 is free to slide within the slot 26 thereby rotating the plane of the bracket member 20. The length of the slot 26 is determined in part by the size of the projection 34 and the desired range of rotation or movement of the bracket member 20 relative to the mounting arrangement 30. In alternative embodiments, the bracket member 20 may comprise the projection 34 and the mounting arrangement 30 comprises the slot 26. In further alternative embodiments, there may comprise a projection positioned so as to limit the range of tilting of the bracket member 20.

Referring back to FIG. 2, each of the connection plates 22 comprise one or more apertures 28. The one or more apertures 28 preferably extend through the connection plates 22. The one or more apertures 28 are positioned so as to align with threaded holes present on most commercially available screens and monitors such as those screens or monitors provided in accordance with the standards as set out by VESA (Video Electronics Standards Association). Alternatively, the apertures 28 may be positioned uniquely for non-standard screens and monitors. In addition, in the preferred embodiment, each of the connection plates 22 comprise at least two of the one or more apertures 28 for aligning with and attaching the screen. However, in further alternative embodiments, each of the connection plates 22 may be provided in separate plates for each of the one or more apertures 28 provided they are each connected at the bracket member 20.

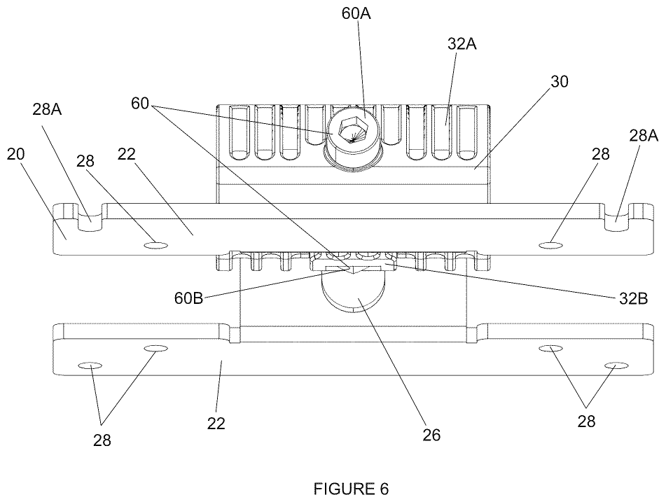

Referring to FIG. 6, there is provided a top view of the assembled screen tilt assembly 10. As can be seen there is provided a clamping mechanism in the form of a bolt and nut arrangement 60. The bolt and nut arrangement 60 provide method for clamping the first clamping member 32A and the second clamping member 32B together. In the preferred embodiment, the second clamping member 32B comprises a recess 64 for receiving a nut 60B of the bolt and nut arrangement 60. The nut 60B of the bolt and nut arrangement 60 may be permanently or removably associated with the second clamping member 32B. The bolt 60A (as seen in FIGS. 4 and 6) of the bolt and nut arrangement 60 may be selectively actuated so as to clamp or release the bracket member 20 from the mounting arrangement 30. The clamping mechanism is advantageously arranged such that the actuating part of the clamping mechanism is preferably accessible and actuatable from the top of the screen tilt mechanism 10. In the preferred embodiment, the bolt 60A is accessible from an upper portion of the first clamping member 32A. Preferably, a head of the bolt 60A is actuatable by a hex or allen key and/or screwdriver for rotating the bolt 60A to clamp/unclamp the bracket member 20. Alternatively, or in addition, the head of the bolt 60A may also be textured such as by knurling or comprise a wingnut to assist the user with imparting torque for actuating the clamping mechanism. In doing so the screen tilt assembly 10 may be adjusted as needed when stowed with multiple medical carts including medical carts which are nestable.

Referring to FIGS. 1, 2 and 6, there is provided additional slots 28A. The additional slots 28A provide for some flexibility in the installation of the screen or monitor to the bracket member 20. The additional slots 28A allow screws to be partially threaded through the at least two of the one or more apertures 28 so as to align the bracket member 20 with the other of the one or more apertures 28. The screen or monitor may then be lifted and positioned on the screen tilt mechanism 10 and positioned thereon whereby the remaining screws and adjustments are made. This may assist with installation by a single person as opposed to requiring multiple people to install and/or irregular or uncomfortable installation practices. In addition, it may also save taking the whole bracket member 20 off the medical cart or other cart which it is mounted when removing the screen or monitor.

The method will now be described with reference to the Figures generally.

To adjust the screen tilt assembly 10 as described above, a user actuates the bolt 60A of the bolt and nut arrangement 60 by rotation so as to unthread at least a portion of the bolt 60A from the nut 60B. Actuating the bolt 60A of the bolt and nut arrangement 60 by rotation in a first direction allows the first clamping member 32A and the second clamping member 32B to unclamp the bracket member 20 from therebetween. Actuating the bolt 60A of the bolt and nut arrangement 60 by rotation in a second direction allows the first clamping member 32A and the second clamping member 32B to clamp the bracket member 20 from therebetween. Rotation in the first direction and second direction merely relates to clockwise and counter clockwise. However, in some instances the bolt and threaded holes may be reverse threaded. Accordingly, they should not be limited to any specific direction of rotation. Furthermore, the person skilled in the art would readily appreciate that there are a number of clamping mechanisms which are known in the art which may be implemented for use with the present invention.

The user may slide, rotate, or otherwise move the bracket member 20 with respect to the mounting arrangement 30 while the first clamping member 30A and the second clamping member 30B are not clamped to the arcuate portion 24 of the bracket member 20. The limits of movement of the bracket member 20 relative to the mounting arrangement 30 will be subject to the arrangement and relative dimensions of the projection(s) 34 and the slot 26. When the user has moved the bracket member 20 such that a screen affixed to the screen tilt assembly 10 is at a desired orientation, the user may then actuate the bolt 60A to clamp the bracket member 22 therebetween the first clamping member 32A and the second clamping member 32B.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. In an aspect, the invention provides a screen tilt assembly for use with medical carts, the screen tilt assembly comprising:
a bracket member comprising at least two spaced apart connection plates arranged in a plane for affixing a screen thereto, the at least two spaced apart connection plates joined by an arcuate portion extending away from the plane; and
a mounting arrangement for connection to a medical cart, the mount arrangement comprising a first clamping member and a second clamping member configured for selectively clamping a section of the arcuate portion of the bracket member therebetween to retain the bracket member in place relative to the mounting arrangement;
wherein the arcuate portion is configured to be selectively moved relative to the mounting arrangement thereby tilting the plane.

2. The screen tilt assembly according to claim 1, wherein the arcuate portion extends in a direction substantially orthogonal to the plane.

3. The screen tilt assembly according to claim 1, wherein the first clamping member and the second clamping member comprise complementary arcuate profiles which conform to the receive therebetween the arcuate portion of the bracket member.

4. The screen tilt assembly according to claim 1, wherein the arcuate member comprises one of a slot or a protrusion and the first clamping member comprises the other one of the slot or the protrusion.

5. The screen tilt assembly according to claim 4, wherein the slot and the protrusion intersect to limit the range of motion of the bracket member relative to the mounting arrangement.

6. The screen tilt assembly according to any one of claim 1, wherein each of the connection plates comprise one or more apertures.

7. The screen tilt assembly according to claim 6, wherein the one or more apertures through each of the connection plates align with one or more threaded holes of a screen.

8. The screen tilt assembly according to any one of claim 1, wherein the mounting arrangement comprises a clamping mechanism for clamping the first clamping mechanism towards the second clamping mechanism.

9. The screen tilt assembly according to claim 8, wherein the clamping mechanism comprises a bolt and nut arrangement.

10. The screen tilt assembly according to claim 9, wherein a bolt head for adjusting the bolt and nut arrangement is accessible from an upper portion of the first clamping member or the second clamping member.

11. The method for adjusting a screen tilt assembly, the method comprising:
providing a screen tilt assembly having a bracket member comprising at least two spaced apart connection plates arranged in a plane for affixing a screen thereto, the at least two spaced apart connection plates joined by an arcuate portion extending away from the plane and a mounting arrangement for connection to a medical cart, the mounting arrangement comprising a first clamping member and a second clamping member configured for selectively clamping a section of the arcuate portion of the bracket member therebetween to retain the bracket member in place relative to the mounting arrangement;
actuating a clamping mechanism to allow the mechanism to move the bracket member relative to the mounting arrangement to a desired orientation and actuating the clamping mechanism to lock the bracket member relative to the mounting arrangement.

12. The method according to claim 11 further comprising loosening a bolt of the clamping mechanism to unclamp the first clamping member from the second clamping member.

13. The method according to claim 11 further comprising tightening the bolt of the clamping mechanism to clamp the first clamping member to or towards the second clamping member.
